(19) United States
(12) Patent Application Publication
(10) Pub. No.: US 2021/0353868 A1
(43) Pub. Date: Nov. 18, 2021

(54) HYDRAULIC SYRINGE

(71) Applicant: CEDARS-SINAI MEDICAL CENTER, Los Angeles, CA (US)

(72) Inventor: Abdul Ali Qaderi, Temecula, CA (US)

(73) Assignee: CEDARS-SINAI MEDICAL CENTER, Los Angeles, CA (US)

(21) Appl. No.: 17/274,241

(22) PCT Filed: Sep. 9, 2019
(86) PCT No.: PCT/US19/50248

§ 371 Date: Mar. 8, 2021

Related U.S. Application Data
(60) Provisional application No. 62/730,678, filed on Sep. 13, 2018.
(60) Provisional application No. 62/805,789, filed on Feb. 14, 2019.

(51) Int. Cl. A61M 5/315; A61M 5/19; A61M 5/31

(52) U.S. Cl. CPC A61M 5/31513; A61M 5/3134; A61M 5/19; A61M 5/31501

(57) ABSTRACT

A positive pressure hydraulic aspiration syringe is provided. The syringe includes a syringe barrel having a proximal end, a distal end and an inner surface extending there between, a terminal orifice having an inner surface extending from the inner surface of the syringe barrel, a plunger having a proximal end, a distal end, and a sidewall extending there between. The plunger is slidably inserted in the syringe barrel such that the sidewall of the plunger forms a moveable seal against the inner surface of the syringe barrel. The syringe also includes a first piston configured to be displaced as the plunger is slidably inserted in the syringe barrel, a second piston mechanically linked to the first piston. Displacement of the first piston causes displacement of the second piston in an opposite direction of the plunger, which creates a negative pressure vacuum at the terminal orifice.

### CROSS REFERENCE TO RELATED APPLICATIONS

This application claims priority under 35 U.S.C. § 119 to U.S. Provisional Application No. 62/730,678, entitled “Hydraulic Syringe,” and filed on Sep. 13, 2018. This application claims priority under 35 U.S.C. § 119 to U.S. Provisional Application No. 62/805,789, entitled “Positive Pressure Hydraulic Aspiration Syringe,” and filed on Feb. 14, 2019. The contents of both applications are hereby incorporated by reference in their entirety.

### FIELD OF THE INVENTION

The present disclosure relates generally to syringe devices, and more specifically, to a positive pressure hydraulic aspiration syringe.

### BACKGROUND

Syringes are widely used to inject, dispense and extract fluids in a controlled fashion. Conventional syringes generally consist of a syringe body having a cylindrical chamber in which a piston is forced to slide. The chamber has an orifice in the end opposite the piston such that if the piston is pushed towards the orifice, fluid is ejected from the chamber into or onto a target. If the piston is forced away from the orifice, fluid at the orifice is vacuumed into the chamber.

As apparent, a source of force is needed to operate a syringe. A non-human driving mechanism, such as an electrical motor, provides this force in automated syringes. Many syringes, however, operate under the manual force of a user. Although typically less sophisticated than automated syringes, manual syringes are widely used because they are inexpensive, easily maneuverable, disposable, and do not require complex and bulky driving mechanisms.

### SUMMARY

A positive pressure hydraulic aspiration syringe is provided. The disclosed syringe includes a syringe barrel having a proximal end, a distal end and an inner surface extending there between. The disclosed syringe also includes a terminal orifice having an inner surface extending from the inner surface of the syringe barrel. The disclosed syringe also includes a plunger having a proximal end, a distal end, and a sidewall extending there between. The plunger is slidably inserted in the syringe barrel such that the sidewall of the plunger forms a moveable seal against the inner surface of the syringe barrel. The disclosed syringe also includes a first piston configured to be displaced as the plunger is slidably inserted in the syringe barrel. The disclosed syringe also includes a second piston mechanically linked to the first piston. Displacement of the first piston causes a displacement of the second piston in an opposite direction of the plunger, which creates a negative pressure vacuum at the terminal orifice.

The disclosed syringe can also include a second chamber having a proximal end, a distal end and an inner surface extending there between. The second chamber is pneumatically connected to the syringe barrel at the distal end. The syringe barrel includes a first diameter, and the second chamber includes a second diameter, which is larger than the first diameter. The syringe barrel is centered with the second chamber. In alternative embodiments, the syringe barrel is adjacent to the second chamber.

The syringe can include a third chamber having a proximal end, a distal end and an inner surface extending there between and connected to the second chamber at the proximal end. The terminal orifice extends from the third chamber. The third chamber includes a third diameter, which is larger than the second diameter. The second chamber is centered with the third chamber. In alternative embodiments, the second chamber is adjacent to the third chamber. The third chamber can have a larger cross section than the syringe barrel and the second chamber.

In some embodiments, hydraulic gas is disposed between the plunger and the first piston. As the plunger is slidably inserted within the syringe barrel, the hydraulic gas displaces the first piston. In alternative embodiments, hydraulic fluid is disposed between the plunger and the first piston. As the plunger is slidably inserted within the syringe barrel, the hydraulic fluid displaces the first piston.

A second exemplary positive pressure hydraulic aspiration syringe is provided. The syringe includes a syringe barrel having a proximal end, a distal end and an inner surface extending there between. The syringe also includes a plunger having a proximal end, a distal end, and a sidewall extending. The plunger is slidably inserted in the syringe barrel such that the sidewall of the plunger forms a moveable seal against the inner surface of the syringe barrel. The syringe also includes a second chamber having a proximal end, a distal end and an inner surface extending there between. The second chamber is connected to the syringe barrel at the distal end. The second chamber includes a first piston. The syringe also includes a third chamber having a proximal end, a distal end and an inner surface extending there between. The third chamber is connected to the second chamber at the proximal end. The third chamber includes a second piston mechanically linked to the first piston of the second chamber.

The second chamber can be pneumatically connected to the syringe barrel. The syringe can also include a terminal orifice having an inner surface extending from the inner surface of the third chamber. In some embodiments, hydraulic gas is disposed between the plunger and the first piston. As the plunger is slidably inserted within the syringe barrel, the hydraulic gas displaces the first piston. Displacement of the first piston can cause displacement of the second piston in an opposite direction of the plunger, creating a negative pressure vacuum in the third chamber. In alternative embodiments, hydraulic fluid is disposed between the plunger and the first piston. As the plunger is slidably inserted within the syringe barrel, the hydraulic fluid displaces the first piston. Displacement of the first piston can cause a displacement of the second piston in an opposite direction of the plunger, creating a negative pressure vacuum in the third chamber.

The syringe barrel includes a first diameter, and the second chamber includes a second diameter, which is larger than the first diameter. The syringe barrel is centered with the second chamber. In alternative embodiments, the syringe barrel is adjacent to the second chamber. The third chamber includes a third diameter, which is larger than the second diameter. The second chamber is centered with the third chamber. In alternative embodiments, the second chamber is adjacent to the third chamber. The third chamber can have a larger cross section than the syringe barrel and the second chamber.

### BRIEF DESCRIPTION OF THE DRAWINGS

In order to describe the manner in which the above-recited disclosure and its advantages and features can be obtained, a more particular description of the principles described above will be rendered by reference to specific examples illustrated in the appended drawings. These drawings depict only example aspects of the disclosure, and are therefore not to be considered as limiting of its scope. These principles are described and explained with additional specificity and detail through the use of the following drawings.

FIG. 1 is a single chamber syringe, in accordance with an implementation of the disclosure;

FIG. 2 is a multi-chamber syringe, in accordance with an implementation of the disclosure;

FIG. 3 is a cross-sectional view of the multi-chamber syringe of FIG. 2, in accordance with an embodiment of the disclosure;

FIG. 4 is an alternative multi-chamber syringe, in accordance with an implementation of the disclosure; and

FIG. 5 is a cross-sectional view of the multi-chamber syringe of FIG. 4, in accordance with an embodiment of the disclosure.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION

The present invention is described with reference to the attached figures, where like reference numerals are used throughout the figures to designate similar or equivalent elements. The figures are not drawn to scale, and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details, or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

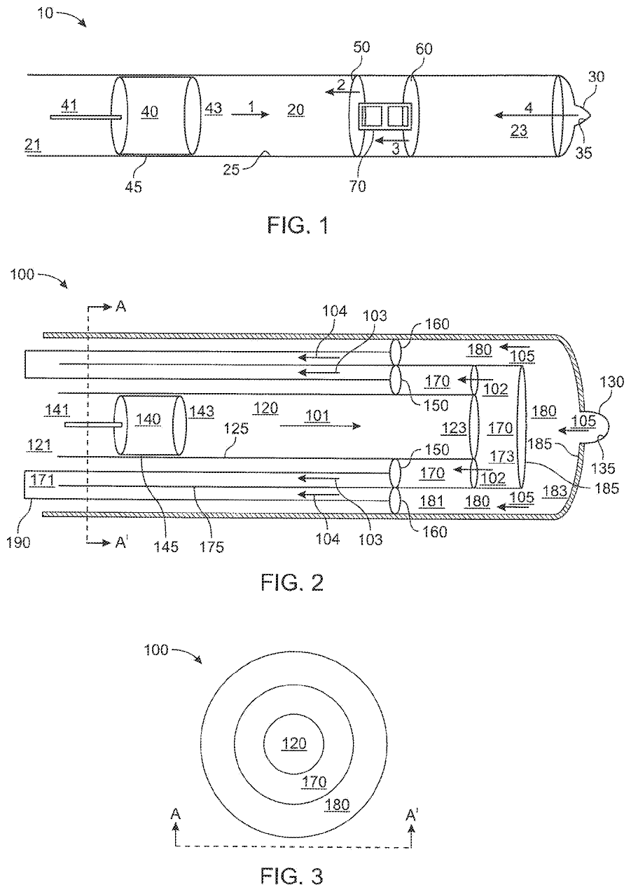

FIG. 1 is a single chamber syringe 10, in accordance with an implementation of the disclosure. The single chamber syringe 10 can include a syringe barrel 20 having a proximal end 23, a distal end 21 and an inner surface 25 extending there between. The single chamber syringe 10 can also include a terminal orifice 30 having an inner surface 35 extending from an inner surface of the single chamber syringe 10. Specifically, the inner surface 35 extends from the inner surface 25 of the syringe barrel 20. The single chamber syringe 10 can also include a plunger 40. The plunger 40 can include a proximal end 43, a distal end 41, and a sidewall 45 extending there between. The plunger 40 is slidably inserted in the syringe barrel 20 in direction 1, such that the sidewall 45 of the plunger 40 forms a moveable seal against the inner surface 25 of the syringe barrel 20. The single chamber syringe 10 also includes a first piston 50 configured to be displaced as the plunger 40 is slidably inserted in the syringe barrel 20. A second piston 60 is mechanically linked 70 to the first piston 50 such that displacement 2 of the first piston 50 causes displacement 3 of the second piston 60 in an opposite direction of the plunger direction 1. This displacement 3 in the second piston 60 creates a negative pressure vacuum 4 at the terminal orifice 30.

In some embodiments, hydraulic gas is disposed between the plunger 40 and the first piston 50 such that as the plunger 40 is slidably inserted within the syringe barrel 20, the hydraulic gas displaces the first piston 50. Alternatively, hydraulic fluid is disposed between the plunger 40 and the first piston 50 such that as the plunger 40 is slidably inserted within the syringe barrel 20, the hydraulic gas displaces the first piston 50.

The single chamber syringe 10 is configured to alter the standard function of a conventional syringe. The configuration of the single chamber syringe 10 is arranged such that a negative pressure vacuum aspirates, draws air/fluid inward, at the terminal orifice 30 with the advancement of the plunger 40. Whereas, in traditional configurations the syringe aspirates when the plunger is withdrawn to create a negative pressure vacuum effect. The manual withdrawal of the plunger is less than desirable in invasive vascular access procedures. Typically, in invasive vascular access procedures, a needle attached to a syringe is inserted into a patient while withdrawing the plunger to create a negative pressure vacuum that will aspirate blood upon entering a vessel. The combination of simultaneously inserting and drawing is incredibly difficult, even for the most trained professionals. The single chamber syringe 10 provides a solution for aspirating at the terminal orifice 30 with positive pressure being applied to the plunger 40. These disclosed embodiments provide procedural ergonomic functionality and anatomic dexterity. Insertion of a needle within the patient while displacing the syringe plunger 40 in the same direction optimizes fine motor movements for improved hand control, accuracy and precision. While the single chamber syringe 10 is provided herein, it should be understood that the functionality disclosed herein can be achieved with multiple combinations of the disclosed elements.

FIG. 2 is a multi-chamber syringe 100, in accordance with an implementation of the disclosure. The multi-chamber syringe 100 can include a syringe barrel 120 having a proximal end 123, a distal end 121 and an inner surface 125 extending there between. The multi-chamber syringe 100 can also include a second chamber 70 having a proximal end 171, a distal end 173 and an inner surface 175 extending there between. The second chamber 170 is pneumatically connected to the syringe barrel 120 at the distal end. In some examples, the second chamber 170 is larger than the syringe barrel 120. Specifically, the diameter of the second chamber 170 can be larger than the diameter of the syringe barrel 120. The multi-chamber syringe 100 can also include a third chamber 180 having a proximal end 181, a distal tapered end 183 and an inner surface 185 extending there between. The third chamber 180 is also connected to the second chamber 170 at the proximal end 181.

The multi-chamber syringe 100 can also include a terminal orifice 130 having an inner surface 135. The terminal orifice 130 extends from the inner surface 185 at the distal tapered end 183 of the third chamber 180. The multi-chamber syringe 100 can also include a plunger 140. The plunger 140 can include a proximal end 143, a distal end 141, and a sidewall 145 extending there between. The plunger 140 is slidably inserted in the syringe barrel 120 in direction 101, such that the sidewall 145 of the plunger 140 forms a moveable seal against the inner surface 125 of the syringe barrel 120. In some embodiments, hydraulic fluid 102 is disposed between the plunger 140 and the first piston 150. As the plunger 140 is slidably inserted within the syringe barrel 120, the hydraulic fluid 102 advances into the second chamber 170 to displace the first piston 150. Alternatively, hydraulic gas 102 is disposed between the plunger 140 and the first piston 150. As the plunger 140 is slidably inserted within the syringe barrel 120, the hydraulic gas 102 advances into the second chamber 170 to displace the first piston 150.

The second chamber 170 includes a first piston 150 configured to be displaced 103 as the plunger 140 is slidably inserted in the syringe barrel 120. A second piston 160 is mechanically linked 190 to the first piston 150 such that displacement 103 of the first piston 150 causes displacement 104 of the second piston 160 in an opposite direction of the plunger direction 101. In some embodiments, the double piston configuration can be linked using a piston rod. The piston rod can be configured such that the displacement of the first piston 150 causes displacement 104 of the second piston 160 in an opposite direction of the plunger direction 101. The displacement 104 in the second piston 160 creates a negative pressure vacuum 105 at the terminal orifice 130. FIG. 2 also includes a cross-sectional line A-A′.

FIG. 3 is a cross-sectional view of the multi-chamber syringe 100 of FIG. 2, in accordance with an embodiment of the disclosure. As indicated above, the second chamber 170 is larger than the syringe barrel 120. Specifically, the diameter of the second chamber 170 is larger than the diameter of the syringe barrel 120. The third chamber 180 is also larger than the second chamber 170 and the syringe barrel 120. Specifically, the diameter of the third chamber 180 is larger than the diameter of the second chamber 170. The diameter of the third chamber 180 is also larger than the diameter of the syringe barrel 120. The syringe barrel 120 is positioned centered to the second chamber 170, which is positioned centered to the third chamber 180.

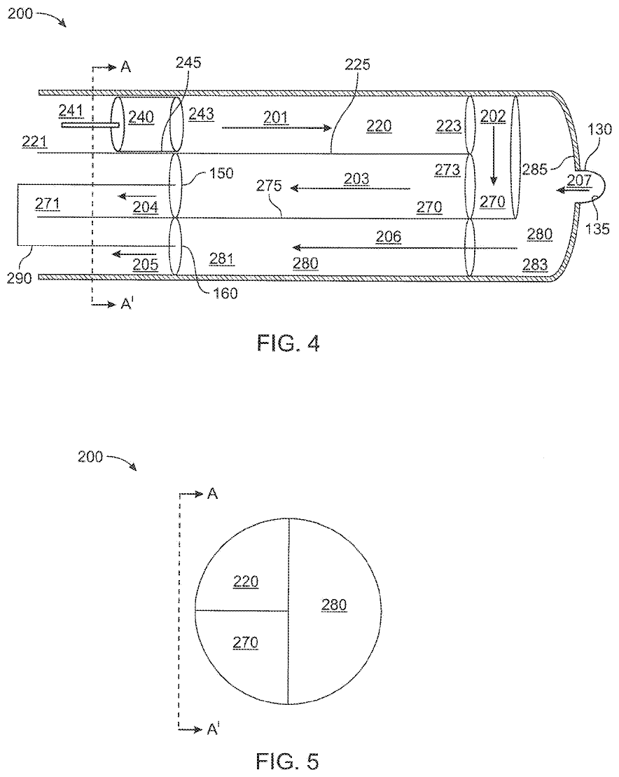

FIG. 4 is an alternative multi-chamber syringe 200, in accordance with an implementation of the disclosure. The multi-chamber syringe 200 can include a syringe barrel 220 having a proximal end 223, a distal end 221 and an inner surface 225 extending there between. The multi-chamber syringe 200 can also include a second chamber 270 having a proximal end 271, a distal end 273 and an inner surface 275 extending there between. The second chamber 270 is adjacent to the syringe barrel 220. Specifically, the distal end 273 of the second chamber 270 is pneumatically connected to the proximal end 223 of the syringe barrel 220. The multi-chamber syringe 200 can also include a third chamber 280 having a proximal end 281, a distal tapered end 283 and an inner surface 285 extending there between. The third chamber 280 is also adjacent to the second chamber 270. Specifically, the proximal end 271 of the second chamber 270 is pneumatically connected to the proximal end 281 of the third chamber 280.

The multi-chamber syringe 200 can also include a terminal orifice 130 having an inner surface 135. The terminal orifice 130 extends from the inner surface 285 at the distal tapered end 283 of the third chamber 280. The multi-chamber syringe 200 can also include a plunger 240. The plunger 240 can include a proximal end 243, a distal end 241, and a sidewall 245 extending there between. The plunger 240 is slidably inserted in the syringe barrel 220 in direction 201, such that the sidewall 245 of the plunger 240 forms a moveable seal against the inner surface 225 of the syringe barrel 220. In some embodiments, hydraulic fluid 202 is disposed between the plunger 240 and the first piston 250. As the plunger 240 is slidably inserted within the syringe barrel 220, the hydraulic fluid 202 advances into the second chamber 270 to displace the first piston 250. Alternatively, hydraulic gas 202 is disposed between the plunger 240 and the first piston 250. As the plunger 240 is slidably inserted within the syringe barrel 220, the hydraulic gas 202 advances into the second chamber 270 to displace the first piston 250.

The second chamber 270 includes a first piston 250 configured to be displaced 204 as the plunger 240 is slidably inserted in the syringe barrel 220. A second piston 260 is mechanically linked 290 to the first piston 250 such that displacement 204 of the first piston 250 causes displacement 205 of the second piston 260 in an opposite direction of the plunger direction 201. The displacement 205 in the second piston 260 drawing in hydraulic gas and/or fluid 206. This displacement 205 thereby creates a negative pressure vacuum 207 at the terminal orifice 130. FIG. 4 also includes a cross-sectional line A-A′.

FIG. 5 is a cross-sectional view of the multi-chamber syringe 200 of FIG. 4, in accordance with an embodiment of the disclosure. As indicated above, the second chamber 270 is adjacent to the syringe barrel 220. The third chamber 280 is also adjacent to the second chamber 270. Specifically, the third chamber 280 has a larger cross section than the syringe barrel 220 and the second chamber 270. While various embodiments are disclosed herein, it should be recognized that any configuration of the disclosed elements such that transfers positive pressure of the plunger 240 into aspiration at the terminal orifice 130.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

While various embodiments of the present invention have been described above, it should be understood that they have been presented by way of example only, and not limitation. Numerous changes to the disclosed embodiments can be made in accordance with the disclosure herein, without departing from the spirit or scope of the invention. Thus, the breadth and scope of the present invention should not be limited by any of the above described embodiments. Rather, the scope of the invention should be defined in accordance with the following claims and their equivalents.

Although the invention has been illustrated and described with respect to one or more implementations, equivalent alterations, and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

What is claimed is:

1. A syringe comprising:
a syringe barrel having a proximal end, a distal end and an inner surface extending there between;
a terminal orifice having an inner surface extending from an inner surface of the syringe;
a plunger having a proximal end, a distal end, and a sidewall, wherein the plunger is slidably inserted in the syringe barrel such that the sidewall of the plunger forms a moveable seal against the inner surface of the syringe barrel;
a first piston configured to be displaced as the plunger is inserted in the syringe barrel; and
a second piston mechanically linked to the first piston such that displacement of the first piston causes displacement of the second piston in an opposite direction of the plunger, creating a negative pressure vacuum at the terminal orifice.

2. The syringe of claim 1, further comprising a second chamber having a proximal end, a distal end and an inner surface extending there between and pneumatically connected to the syringe barrel at the distal end.

3. The syringe of claim 2, wherein the syringe barrel comprises a first diameter, and the second chamber comprises a second diameter, such that the second diameter is larger than the first diameter and the syringe barrel is centered with the second chamber.

4. The syringe of claim 2, wherein the syringe barrel is adjacent to the second chamber.

5. The syringe of claim 2, further comprising a third chamber having a proximal end, a distal tapered end and an inner surface extending there between and connected to the second chamber at the proximal end, wherein the terminal orifice extends from the distal tapered end of the third chamber.

6. The syringe of claim 5, wherein the third chamber comprises a third diameter, and the third diameter is larger than the second diameter and the second chamber is centered with the third chamber.

7. The syringe of claim 5, wherein the second chamber is adjacent to the third chamber.

8. The syringe of claim 7, wherein the third chamber has a larger cross section than the syringe barrel and the second chamber.

9. The syringe of claim 1, wherein at least a portion of hydraulic gas is disposed between the plunger and the first piston such that as the plunger is slidably inserted within the syringe barrel, the hydraulic gas displaces the first piston.

10. The syringe of claim 1, wherein at least a portion of hydraulic fluid is disposed between the plunger and the first piston such that as the plunger is slidably inserted within the syringe barrel, the hydraulic fluid displaces the first piston.

11. A syringe comprising:
a syringe barrel having a proximal end, a distal end and an inner surface extending there between;
a plunger having a proximal end, a distal end, and a sidewall extending, wherein the plunger is slidably inserted in the syringe barrel such that the sidewall of the plunger forms a moveable seal against the inner surface of the syringe barrel;
a second chamber having a proximal end, a distal end and an inner surface extending there between and connected to the syringe barrel at the distal end, wherein the second chamber comprises a first piston; and
a third chamber having a proximal end, a distal tapered end and an inner surface extending there between and connected to the second chamber at the proximal end, comprising a second piston mechanically linked to the first piston of the second chamber.

12. The syringe of claim 11, wherein the second chamber is pneumatically connected to the syringe barrel.

13. The syringe of claim 11, further comprising a terminal orifice extending from the distal tapered end of the third chamber.

14. The syringe of claim 11, wherein at least a portion of hydraulic gas is disposed between the plunger and the first piston such that as the plunger is slidably inserted within the syringe barrel, the hydraulic gas displaces the first piston.

15. The syringe of claim 14, wherein displacement of the first piston causes displacement of the second piston in an opposite direction of the plunger, creating a negative pressure vacuum in the third chamber.

16. The syringe of claim 11, wherein at least a portion of hydraulic fluid is disposed between the plunger and the first piston such that as the plunger is slidably inserted within the syringe barrel, the hydraulic fluid displaces the first piston.

17. The syringe of claim 16, wherein displacement of the first piston causes a displacement of the second piston in an opposite direction of the plunger, creating a negative pressure vacuum in the third chamber.

18. The syringe of claim 11, wherein the syringe barrel comprises a first diameter, and the second chamber comprises a second diameter, such that the second diameter is larger than the first diameter and the syringe barrel is centered with the second chamber.

19. The syringe of claim 18, wherein the third chamber comprises a third diameter, and the third diameter is larger than the second diameter and the second chamber is centered with the third chamber.

20. The syringe of claim 1, wherein the syringe barrel is adjacent to the second chamber, the second chamber is adjacent to the third chamber, and the third chamber has a larger cross section than the syringe barrel and the second chamber.
